SEQUENCE LISTING (4 sequences)

SEQ ID NO: 1 - protein, 11 aa, Artificial Sequence
  Synthetic construct
TALDWSWLQTE

SEQ ID NO: 2 - protein, 28 aa, Artificial Sequence
  Synthetic construct
DRQIKIWFQNRRMKWKKTALDWSWLQTE

SEQ ID NO: 3 - protein, 6 aa, Artificial Sequence
  Synthetic construct
LDWSWL

SEQ ID NO: 4 - protein, 22 aa, Artificial Sequence
  Synthetic construct
DRQIKWFQNRRMKWKKLDWSWL

(19) United States
(12) Patent Application Publication
(10) Pub. No.: US 2024/0156902 A1
(43) Pub. Date: May 16, 2024

(54) METHODS OF TREATING NEURODEGENERATIVE DISORDERS WITH INTRANASAL NF-KAPPAB ESSENTIAL MODIFIER (NEMO)-BINDING DOMAIN (NBD) PEPTIDE

(71) Applicant: Rush University Medical Center, Chicago, IL (US)

(72) Inventor: Kalipada Pahan, Skokie, IL (US)

(21) Appl. No.: 18/550,859

(22) PCT Filed: Mar. 16, 2022
(86) PCT No.: PCT/US2022/020506

§ 371 Date: Sep. 15, 2023

Related U.S. Application Data
(60) Provisional application No. 63/161,490, filed on Mar. 16, 2021.

(51) Int. Cl. A61K 38/17; A61K 9/00; A61P 25/28

(52) U.S. Cl. CPC A61K 38/1709; A61K 9/0043; A61P 25/28

(57) ABSTRACT

The present disclosure generally relates to pharmaceutical compositions useful for the treatment of diseases and disorders. More particularly, the disclosure relates to pharmaceutical compositions comprising peptides that selectively inhibit NF-κB activation control or inhibit alpha(α)-synucleinopathy and neuronal loss in neurodegenerative diseases in which α-synuclein and/or NF-κB play a role in disease pathogenesis. The pharmaceutical compositions useful for the invention are preferably administered intranasally.

### CROSS-REFERENCE TO RELATED APPLICATIONS

The present application claims the benefit of U.S. Provisional Patent Application No. 63/161,490, filed Mar. 16, 2021, the contents of which are incorporated into the present application in their entirety.

### REFERENCE TO GOVERNMENT GRANTS

This invention was made with government support under grant number NS108025 awarded by National Institutes of Health. The government has certain rights.

### FIELD OF THE INVENTION

The present disclosure generally relates to pharmaceutical compositions useful for the treatment of diseases and disorders. More particularly, the disclosure relates to pharmaceutical compositions comprising peptides that selectively inhibit NF-κB activation control or inhibit alpha(α)-synucleinopathy and neuronal loss in neurodegenerative diseases in which α-synuclein and/or NF-κB play a role in disease pathogenesis

### SEQUENCE LISTING

The instant application contains a Sequence Listing which has been submitted electronically in ASCII format and is hereby incorporated by reference in its entirety. The ASCII copy, created on Mar. 15, 2022, is named R642_SEQ_LISTING_ST25.txt and is 1 KB in size.

### BACKGROUND

Although Parkinson's disease (PD) is the second most common neurodegenerative disorder, despite intense investigations, to date, no effective therapy is available to stop its onset or halt its progression. Previous studies have shown ability of peptide corresponding to the NF-κB essential modifier-binding domain (NBD) of IkB kinase α (IKKα) or IKKbeta to prevent nigrostriatal degeneration in the 1-methyl-4-phenyl-1,2,3,6-tetrahydropyridine (MPTP) mouse model of PD and establish a role for NF-κB in human parkinsonism. It was previously found that NF-κB was activated within the substantia nigra pars compacta of PD patients and MPTP-intoxicated mice. However, i.p. injection of wild-type NBD peptide, but not mutated NBD peptide, reduced nigral activation of NF-κB, suppressed nigral microglial activation, protected both the nigrostriatal axis and neurotransmitters, and improved motor functions in MPTP-intoxicated mice. These studies suggested that selective inhibition of NF-κB activation by NBD peptide may be of therapeutic benefit for PD patients. See Ghosh et al., “Selective inhibition of NF-κB activation prevents dopaminergic neuronal loss in a mouse model of Parkinson's disease,” PNAS (2007), 104: pp. 18754-18759.

Furthermore, since neuroinflammation plays an important role in the pathogenesis of PD and NF-κB, a proinflammatory transcription factor, participates in the transcription of many proinflammatory molecules, previous studies have evaluated the ability of a NBD peptide to protect dopaminergic neurons in hemiparkinsonian monkeys. It was found that that NF-κB was activated within the substantia nigra pars compacta of 1-methyl-4-phenyl-1,2,3,6-tetrahydropyridine (MPTP)-intoxicated hemiparkinsonian monkeys. However, intramuscular injection of wild type NBD (wtNBD) peptide (but not the mutated form) reduced nigral activation of NF-κB and expression of inducible nitric oxide synthase, protected both the nigrostriatal axis and neurotransmitters, and improved motor functions in hemiparkinsonian monkeys. See Mondal et al., “Testing NF-κB-based therapy in hemiparkinsonian monkeys,” (2012) J Neuroimmune Pharmacol. 7: pp. 544-556.

One of the pathologic hallmarks of PD is the presence of Lewy bodies (LBs) containing aggregated α-synuclein (α-syn). In addition to PD, prion-like spreading of pathological α-syn aggregates in the brain and associated neuropathology in α-synucleinopathy area also observed in multiple system atrophy (MSA), and dementia with Lewy bodies (DLB). See for example Bae et al., “Glucocerebrosidase depletion enhances cell-to-cell transmission of α-synuclein,” (2014), Nat Commun 5, p. 4755; Lee et al., “Extracellular α-synuclein-a novel and crucial factor in Lewy body diseases,” (2014), Nat Rev Neurol 10: pp. 92-98; Luk et al., “Pathological α-synuclein transmission initiates Parkinson-like neurodegeneration in nontransgenic mice,” (2012), Science 338: pp. 949-953.

However, mechanisms by which α-syn spreading occurs leading to loss of neurons in the brain are poorly understood. Furthermore, there no studies describing the intranasal use of intranasal low dose NBD peptide may be beneficial for MSA, DLB and PD as well as other neurodegenerative diseases such as multiple sclerosis (MS), optic neuritis (ON), Huntington disease (HD), Amyotrophic lateral sclerosis (ALS) in which α-syn and/or microglial activation play a role in disease pathogenesis. One of the pathologic hallmarks of PD is the presence of Lewy bodies (LBs) containing aggregated α-synuclein (α-syn). Lowering the deposition of aggregated α-syn from the brain parenchyma is expected to reduce the development and progression of not only sporadic and familial PD, but also dementia with Lewy bodies (DLB) and multiple system atrophy (MSA)

The inventor addresses this need herein by demonstrating that selective inhibition of NF-κB activation by intranasal wild type NEMO-binding domain (wtNBD) peptide decreases α-syn spreading, protected dopaminergic neurons and improvement in the preformed α-syn fibril (PFF)-seeded mouse model of α-synucleinopathy. Therefore, α-syn spreading and associated loss of neurons depends on NF-κB and intranasal wtNBD peptide at a very low dose can provide new therapeutic options to control α-synucleinopathy and neuronal loss in MSA, DLB and PD and other neurodegenerative diseases in which α-syn and/or NF-κB play a role in disease pathogenesis.

### SUMMARY OF THE DISCLOSURE

The inventors have discovered methods and pharmaceutical compositions and/or formulations useful for the treatment of neurodegenerative and disorders involving α-synucleinopathy. More particularly, the present disclosure relates to methods and pharmaceutical compositions and/or formulations comprising agents that inhibit NF-κB activation. Even more particularly, the present disclosure provides methods and compositions comprising a NEMO-binding domain (NBD) peptide to slow or inhibit the progression of neurodegenerative and disorders involving α-synucleinopathy.

In some embodiments, the pharmaceutical composition comprises an agent that inhibits NF-κB activation where the agent is a wild-type NEMO-binding domain (wtNBD) peptide. In other embodiments, the wtNBD peptide contains the Antennapedia homeodomain or similar petide sequence to promote entrance into the cells. In still other embodiments, the wtNBD peptide contains the inhibitor of nuclear factor kappa-B kinase subunit beta (IKKβ) amino acid.

In any embodiments, the pharmaceutical composition is formulated together with a pharmaceutically acceptable carrier or excipient.

In still other embodiments, the pharmaceutical composition is preferably administered intranasally.

In any embodiments, the pharmaceutical compositions are used to treat or inhibit the progression or spreading of α-syn, more particularly, disorders that involve α-synucleinopathy. More particularly, the pharmaceutical compositions are used to treat or inhibit the progression or spreading of α-syn in disorders including multiple system atrophy (MSA), dementia with Lewy bodies (DLB), PD, multiple sclerosis (MS), optic neuritis (ON), Huntington disease (HD), Amyotrophic lateral sclerosis (ALS), or any disorder in which microglial activation may play a role in disease pathogenesis.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1A shows the stereotaxic placement of the preformed α-syn fibril (PFF) in a mouse model of Lewy body diseases. FIG. 1B shows the treatment parameters of 3 months old A53T transgenic mice injected in a stereotaxic frame with 5 μg of PFF in both the hemispheres of brain. Following 2 months of surgery, animals received wild type NBD (wtNBD) at a dose of 0.1 mg/kg body weight/day intranasally. After 1 month of wtNBD treatment, behavioral analyses were performed followed by immunohistochemistry and different biochemical experiments.

FIG. 2A and FIG. 2B show the results following intranasal administration of wtNBD peptide inhibiting NF-κB activation in the nigra of PFF-seeded mouse model of Lewy body diseases. A53T transgenic mice were seeded with PFF bilaterally and following 2 months of brain surgery, animals were given intranasal administration of 0.1 mg/kg wtNBD peptide daily. Activation of NF-κB in nigra was monitored by evaluating the level of acetylated (K310) p65 in Ibal+ve microglia in different groups of mice. Marked up-regulation of microglial acetylated p65 level in the SN of PFF-seeded mice was found compared to the PBS-injected mice (FIG. 2A and FIG. 2B). However, acetylated p65 level significantly decreased in wtNBD-treated mice brain (FIG. 2A and FIG. 2B). Statistical significance was determined by one-way ANOVA followed by Tukey's multiple comparison tests. *p<0.05, ***p<0.001 indicate significance compared to respective groups. Values are given as mean±SEM (n=4 per group).

FIG. 3A-H shows the results following intranasal administration of wtNBD peptide inhibiting α-syn spreading from striatum to nigra and motor cortex in PFF-seeded mouse model of Lewy body diseases. Propagation of α-syn in PFF-seeded A53T mouse brain was monitored in SN by pSyn129 immunostaining and relative intensity measurement (FIG. 3A, FIG. 3B) and also by immunoblotting total α-syn in Triton X-100 soluble and insoluble fractions (FIG. 3C, FIG. 3D). The ratio of α-syn to actin is shown in the diagrams (FIG. 3E, FIG. 3F). Level of pSyn129 in motor cortex was assessed by immunohistochemistry (FIG. 3G, FIG. 3H). Two sections from each brain were used for immunostaining and pSyn129 specific intensity was analysed by Fiji. One-way ANOVA followed by Tukey's multiple comparison tests was conducted for statistical analyses. **p<0.01, ***p<0.001 indicate significance compared to respective groups. Values are given as mean±SEM (n=4 animals per group).

FIG. 4A-L depicts the results following intranasal administration of wtNBD peptide reducing Parkinsonian pathology in PFF-seeded mouse model of Lewy body diseases. PFF-seeded A53T animals were given intranasal administration of 0.1 mg/kg wtNBD peptide daily for one month Parkinsonian pathology was evaluated by TH immunohistochemistry of nigral sections (FIG. 4A), immunoblotting of total TH level in SN (FIG. 4B and FIG. 4C), assessing striatal level of dopamine (DA), and its metabolites 3,4-dihydroxyphenyl acetate (DOPAC), homovanillic acid (HVA) (FIG. 4D-F). Behavioral analyses of animals were performed by open field test (FIG. 4G), where movement parameters such as distance (FIG. 4H), velocity (FIG. 4I), cumulative duration (FIG. 4J) and rearings (FIG. 4K) were recorded. Feet movement was analysed by rotarod test (FIG. 4L). Statistical significance was determined by one-way ANOVA followed by Tukey's multiple comparison tests. *p<0.05, **p<0.01, ***p<0.001 indicate significance compared to respective groups. Values are given as mean±SEM (n=4 per group).

### DETAILED DESCRIPTION

Throughout this disclosure, various quantities, such as amounts, sizes, dimensions, proportions, and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “amino acid” refers, in particular, to any one of the 20 standard proteinogenic a-amino acids (i.e., Ala, Arg, Asn, Asp, Cys, Glu, Gin, Gly, His, He, Leu, Lys, Met, Phe, Pro, Ser, Thr, Trp, Tyr, and Val) but also to non-proteinogenic and/or non-standard a-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyc! opentylaianine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., y-aminobutyric acid, isoglutamine, or statine) and/or δ-amino acids as well as any other compound comprising at least one carboxylic acid group and at least one amino group. Unless defined otherwise, an “amino acid” preferably refers to an a-amino acid, more preferably to any one of the 20 standard proteinogenic a-amino acids (which can be present as the L-isomer or the D-isomer, and are preferably present as the L-isomer).

The terms “peptide” and “polypeptide,” are used herein interchangeably and refer to a polymer of two or more amino acids linked via amide bonds that are formed between an amino group of one amino acid and a carboxyl group of another amino acid. the term peptide or polypeptide, it is meant to include the peptide or polypeptide itself, as well as any physiologically acceptable salts thereof, or any chemically modification made thereto, which would be apparent or known to a person of ordinary skill in the art. The amino acids comprised in the peptide or polypeptide, which are also referred to as amino acid residues, may be selected from the 20 standard proteinogenic a-amino acids (i.e., Ala, Arg, Asn, Asp, Cys, Glu, Gin, Gly, His, lie, Leu, Lys, Met, Phe, Pro, Ser, Thr, Trp, Tyr, and Val) but also from non-proteinogenic and/or non-standard a-amino acids (such as, e.g., ornithine, citrulline, homolysine, pyrrolysine, 4-hydroxyproline, α-methylalanine (i.e., 2-aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β-amino acids (e.g., β-alanine), γ-amino acids (e.g., γ-aminobutyric acid, isogiutamine, or statine) and δ-amino acids. Preferably, the amino acid residues comprised in the peptide or polypeptide are selected from a-amino acids, more preferably from the 20 standard proteinogenic a-amino acids (which can be present as the L-isomer or the D-isomer, and are preferably all present as the L-isomer). The peptide or polypeptide may be unmodified or may be modified, e.g., at its N-terminus, at its C-terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts PG & Greene TW, “Greene's protective groups in organic synthesis,” John Wiley & Sons, 2006. Such modifications may also include the covalent attachment of one or more polyethylene glycol (PEG) chains (forming a PEGylated peptide or polypeptide), the glycosylation and/or the acylation with one or more fatty acids (e.g., one or more C₈₋₃₀ alkanoic or alkenoic acids; forming a fatty acid acyiated peptide or polypeptide). Moreover, such modified peptide or proteins may also include peptidomimetics, provided that they contain at least two amino acids that are linked via an amide bond (formed between an amino group of one amino acid and a carboxyl group of another amino acid). The amino acid residues comprised in the peptide or polypeptide may, e.g., be present as a linear molecular chain (forming a linear peptide or protein) or may form one or more rings (corresponding to a cyclic peptide or polypeptide). The peptide or polypeptide may also form oligomers consisting of two or more identical or different molecules.

The term “identity” refers to the overall relatedness between polymeric molecules, e.g., between peptides or polypeptides. Methods for the calculation of a percent identity as between two provided polypeptide sequences are known. Calculation of the percent identity of two polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

To calculate percent identity, the sequences being compared are typically aligned in a way that gives the largest match between the sequences. One example of an algorithm available for comparison of amino acid or nucleic acid sequences, comprising those available in commercial computer programs is BLASTN for nucleotide sequences and BLASTP, gapped BLAST, and PSI-BLAST for amino acid sequences. Exemplary programs are described in Altschul, et al., “Basic local alignment search tool,” J. Mol. Biol., 215(3): 403-410, 1990; Altschul, et al., “Methods in Enzymology;” Altschul, et al., “Gapped BLAST and PSI-BLAST: a new generation of protein database search programs,” Nucleic Acids Res. 25:3389-3402, 1997; Baxevanis, et al., “Bioinformatics: A Practical Guide to the Analysis of Genes and Proteins,” Wiley, 1998; and Misener, et al., (eds.), Bioinformatics Methods and Protocols (Methods in Molecular Biology, Vol. 132), Humana Press, 1999. In addition to identifying similar sequences, the programs mentioned above generally provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues (e.g., 85-90%, 85-95%, 85-100%, 90-95%, 90-100%, or 95-100%). In some embodiments, the relevant stretch is a complete sequence.

The term “subject” or “patient” as used herein, refers to a mammal, in some aspects a human.

A “therapeutically effective amount,” “effective dose,” “effective amount,” or “therapeutically effective dosage” of a therapeutic agent, e.g., a peptide, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The therapeutic agent may inhibit (lessen the severity of or eliminate the occurrence of) and/or prevent a disorder, and/or any one of the symptoms of the disorder. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

“Treating,” “treat”, or “treatment” within the context of the instant disclosure, means an alleviation of symptoms associated with a disorder or disease, or halt of further progression or worsening of those symptoms, or prevention or prophylaxis of the disease or disorder. For example, within the context of this disclosure, successful treatment may include an alleviation of symptoms related to a neurodegenerative disorder as those described herein. The treatment may include administering an effective amount of a peptide to the subject that results in an alleviation of symptoms associated with a disorder or disease, or halt of further progression or worsening of those symptoms, or prevention or prophylaxis of the disease or disorder.

The present disclosure is based on the discovery that low dose intranasal administration of NF-κB essential modifier (NEMO)-binding domain (NBD) peptide which is a specific inhibitor of NF-κB activation, alone or in a pharmaceutical composition, may be beneficial for treating or inhibiting the progression or spreading of α-syn, more particularly, disorders that involve α-synucleinopathy. More particularly, the pharmaceutical compositions are used to treat or inhibit the progression or spreading of α-syn in disorders including multiple system atrophy (MSA), dementia with Lewy bodies (DLB), PD, multiple sclerosis (MS), optic neuritis (ON), Huntington disease (HD), Amyotrophic lateral sclerosis (ALS), or any disorder in which microglial activation may play a role in disease pathogenesis.

Currently, no therapies are available for α-synucleinopathy. Since microglial activation plays an important role in different neurodegenerative diseases and activation of NF-κB is needed for microglial inflammation, the inventor investigated the role of NF-κB in α-syn spreading and associated pathology seen in the brain of MSA, DLB and PD patients. NF-κB essential modifier (NEMO)-binding domain (NBD) peptide is a specific inhibitor of NF-κB activation. Therefore, the effect of intranasal wtNBD on α-syn spreading and associated neuronal death was examined in preformed α-syn fibril (PFF)-seeded mouse model of α-synucleinopathy. It was discovered that intranasal wtNBD peptide at a very low dose (0.1 mg/kg body wt/d) decreased α-syn spreading, protected dopaminergic neurons and improved locomotor activities in PFF-seeded A53T transgenic mice.

The treatment comprises administering an effective amount of a pharmaceutical composition comprising the NF-κB essential modifier (NEMO)-binding domain (NBD) peptide which is a specific inhibitor of NF-κB activation. In a preferred embodiment, the NBD peptide is administered intranasally to a patient in need thereof. The treatment may be administered one time per day. In some aspects, the treatment may be administered two times per day, three times per day, or more than three times per day.

The NBD peptide may be formulated for administration. Methods of formulation are well known in the art (see, for example, Remington: The Science and Practice of Pharmacy, Mack Publishing Company, Easton, Pa., 19th Edition (1995)). Pharmaceutical compositions for use in accordance with the present disclosure can be in the form of sterile, non-pyrogenic intranasal or other liquid solutions or suspensions, coated capsules, lyophilized powders, or other forms known in the art.

Pharmaceutically Acceptable Carrier

As used herein, the term “pharmaceutically acceptable carrier” means a non-toxic, inert solid, semi-solid or liquid filler, diluent, encapsulating material or formulation auxiliary of any type. In the treatment methods contemplated by the present disclosure, the NBD peptide may be used alone or in compositions together with a pharmaceutically acceptable carrier or excipient, such as saline. For example, an oral dosage form composition may comprise NBD peptide in addition to a pharmaceutically acceptable carrier. An inhalation dosage form composition may an NBD peptide in addition to a pharmaceutically acceptable carrier. A composition for buccal administration may comprise an NBD peptide in addition to a pharmaceutically acceptable carrier. A composition for nasal administration may comprise an NBD peptide in addition to a pharmaceutically acceptable carrier. Further, if a transdermal patch is used as the method of administering the NBD peptide to the patient, the transdermal patch may comprise the NBD peptide in addition to a pharmaceutical acceptable carrier.

Some examples of materials which can serve as pharmaceutically acceptable carriers are sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil; safflower oil; sesame oil; olive oil; corn oil and soybean oil; glycols, such as propylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol, and phosphate buffer solutions, as well as other non-toxic compatible lubricants, such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, the contents of which are expressly incorporated herein by reference.

Oral Dosage Forms

In certain embodiments, the NBD peptide may be orally administered to be ingested by humans and other animals. Solid dosage forms for oral administration include, as illustrative but non-limiting examples, capsules, tablets, pills, powders, thin films and granules. In solid dosage forms, the active compound may be mixed with at least one inert, pharmaceutically acceptable excipient or carrier, as described in more detail below.

As illustrative, non-limiting examples, an oral dosage form of the presently disclosed pharmaceutical composition may be mixed with about 0.1% to about 1%, such as about 0.5%, methyl cellulose.

A pharmaceutical composition according to the present disclosure for intranasal administration may be mixed with about 1 to about 10 μl, such as about 5 μl, of saline. A pharmaceutical composition according to the present disclosure for nebulization may be solubilized in about 100 to about 300 μl saline, such as about 200 μl saline.

Stabilizers

A composition, formulation, or dosage form herein may further comprise NBD peptide stabilizers. As used herein, a NBD peptide stabilizer is a substance that extends the time before which the NBD peptide composition is converted to a salt in the environment in which the formulation or dosage form is administered, in comparison to the conversion in its absence. Non-limiting examples of stabilizers include phosphatidyl choline, phosphatidyl inositol, phosphatidyl ethanolamine, or other phospholipids. A composition, formulation, or dosage form further comprising one or more stabilizers may be administered in any one of the methods herein. A NBD peptide stabilizer may be present in an amount of about 50 mg to about 1000 mg in a composition, formulation, or dosage form herein. In some embodiments, the stabilizer may be present in an amount ranging from about 50 mg to about 500 mg or about 50 mg to about 100 mg.

As an additional example, in addition to an NBD peptide and/or a pharmaceutically acceptable carrier, an inhalation dosage form composition may comprise one or more stabilizers. A stabilizer in an inhalation dosage from may be present in an amount of about 50 mg to about 1000 mg. In some embodiments, the stabilizer may be present in an amount ranging from about 50 mg to about 500 mg, about 50 mg to about 100 mg, or less than about 50 mg.

As a further example, in addition to an NBD peptide and/or a pharmaceutically acceptable carrier, a composition for buccal administration may comprise one or more stabilizers. A stabilizer in a composition for buccal administration may be present in an amount of about 50 mg to about 1000 mg. In some embodiments, the stabilizer may be present in an amount ranging from about 50 mg to about 500 mg, about 50 mg to about 100 mg, or less than about 50 mg.

In addition to an NBD peptide and/or a pharmaceutically acceptable carrier, a transdermal patch may comprise one or more stabilizers. A stabilizer in a composition for transdermal administration may be present in an amount of about 50 mg to about 1000 mg. In some embodiments, the stabilizer may be present in an amount ranging from about 50 mg to about 500 mg, about 50 mg to about 100 mg, or less than about 50 mg. As is commonly understood in the art, a transdermal patch is an adhesive patch that is placed on the skin of a patient. The patch comprises a composition/medication and delivers the composition/medication to the patient through the skin.

Intranasal Compositions

In preferred embodiments, the pharmaceutical composition may be administered to a patient as nasal drop (intranasally) or using a nebulization technique. A nebulizer may be used to change a liquid solution of a pharmaceutical composition into a fine mist that may be inhaled by a patient. The inventor determined numerous benefits of these techniques.

For example, the dosage of the pharmaceutical composition can be significantly decreased when either nasal drop or nebulization is used as the delivery method. In some instances, the dosage may be reduced by about one tenth or one twentieth as compared to, for example, injections, oral administration/ingestion of a liquid solution or oral administration/ingestion of a pill. Moreover, using a nebulization technique or nasal drop bypasses the digestive system whereas ingesting a pill or liquid solution of a pharmaceutical composition sends the composition to the digestive system. Finally, using either a nasal drop or nebulization technique allows the pharmaceutical composition to travel from the olfactory bulb directly to the brain.

In some embodiments, the nebulized pharmaceutical composition may be inhaled through one or both of the mouth or the nasal passage. Without being bound to any theory, it is believed that nasal administration of the composition can take advantage of “nose-to-brain” (N2B) transport systems in which several possibilities exist for bypassing the blood-brain-barrier for direct delivery to the brain. These include the draining of drugs absorbed in the nasal mucosa into the sinus and eventually to the carotid artery, where a “counter-current transfer” from venous blood to the brain may occur. Lymphatic drainage into the perivascular space from the olfactory trigeminal nerves between the central nervous system (CNS) have also been postulated as the mechanism of N2B transport.

Nebulizers are known in the art and the invention of the present disclosure can be used in connection with any nebulizer. For example, the pharmaceutical composition disclosed herein may be nebulized with an inhaler or a Buxco® Inhalation Tower All-In-One Controller.

Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells. Illustrative, non-limiting examples of coatings and shells include enteric coatings and other coatings/shells well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that may be used include, but are not limited to, polymeric substances and waxes.

The active compounds can also be in micro-encapsulated form with one or more excipients as noted above. The solid dosage forms of tablets, dragees, capsules, pills, and granules can be prepared with coatings and shells. The coatings or shells may be, but are not limited to, enteric coatings, release-controlling coatings and other coatings in the pharmaceutical formulating art. In solid dosage forms, the active compound may be admixed with at least one inert diluent. The inert diluent may include, but is not limited to, one or more of, sucrose, lactose or starch. Dosage forms may also comprise additional substances other than inert diluents. The additional substances may be, but are not limited to, tableting lubricants and other tableting aids. The tableting lubricants and other aids may be, but are not limited to, magnesium stearate and microcrystalline cellulose. In the case of capsules, tablets and pills, for example, the dosage forms may also comprise buffering agents. They may comprise opacifying agents. They may be of a composition that releases the active ingredient(s) only, or preferentially, in a certain part of the intestinal tract. The release may be in a delayed manner. Examples of embedding compositions that can be used include, but are not limited to, polymeric substances and waxes.

Liquid Dosage Forms

Liquid dosage forms for oral administration include, but are not limited to, pharmaceutically acceptable emulsions, microemulsions, solutions, suspensions, syrups and elixirs. In addition to the active compounds, the liquid dosage forms may comprise one or more inert diluents. The inert diluents may be selected from those commonly used in the art. Illustrative, non-limiting examples of inert diluents include water or other solvents, solubilizing agents and emulsifiers including, but not limited to, ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The oral compositions may comprise one or more adjuvants. Illustrative, non-limiting examples of adjuvants include wetting agents, emulsifying and suspending agents, sweetening, flavoring, and perfuming agents.

The amount of carrier in a composition disclosed herein is not particularly limited. As an example, for a liquid oral treatment composition, the composition may comprise from about 0.1% carrier to about 1% carrier, such as about 0.5% methyl cellulose. In some embodiments, for intranasal administration, the composition may comprise from about 1 μl to about 10 μl of the carrier, such as about 5 μl saline. In some embodiments, for nebulization, the composition may comprise from about 50 μl to about 500 μl of the carrier, such as about 100 μl, about 200 μl or about 300 μl saline.

“Effective or Therapeutic Amount”

Effective or therapeutic amounts of the compositions of this disclosure include any amount sufficient to inhibit (e.g., slow or stop) the progression of a neurodegenerative disorder. In some embodiments, effective amounts of the compositions include any amount sufficient to inhibit (e.g., slow or stop) the deterioration of a locomotor activity of a patient. In some embodiments, effective amounts of the compositions include any amount sufficient to improve a locomotor activity of a patient. In some embodiments, effective amounts of the compositions include any amount sufficient to reduce a level of aggregated α-synuclein in the brain. In some embodiments, effective amounts of the compositions include any amount sufficient to reduce glial cell activation.

The amount of active ingredient (a NBD peptide) that may be combined with the optional carrier materials to produce a single dosage form may vary depending upon the host treated and the particular mode of administration. The specific dose level for any particular patient may depend upon a variety of factors including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

In accordance with certain methods of treatment disclosed in the present application, progression of various disorders is slowed or stopped in a patient (a patient may be a human, a lower mammal, or a warm-blooded animal), by administering to the patient an effective amount of the a NBD peptide in such amounts, and for such time as is necessary, to achieve the desired result. An amount of a compound that is effective to slow or stop the progression of a disease or disorder may refer to a sufficient amount of the compound to treat the disease or disorder at a reasonable benefit/risk ratio applicable to any medical treatment.

The total daily usage of the compounds and compositions of the present disclosure may be decided by the attending physician within the scope of sound medical judgment. The specific therapeutically effective dose level for any particular patient may depend upon a variety of factors including the disease or disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; and drugs used in combination or coincidental with the specific compound employed.

The “effective amount” or dose of a compound of the present disclosure, such as a NBD peptide, to be administered to warm-blooded animals (e.g., humans) may vary depending upon the disorder to be treated.

However, if intranasal administration is used as the method of administering the pharmaceutical composition, the inventor determined that in some embodiments, the amount administered to the patient may be from about 1 mg/kg body weight per day to about 25 mg/kg body weight per day. In some embodiments, the effective amount may be from about 1 mg/kg body weight per day to about 15 mg/kg body weight per day, from about 1 mg/kg body weight per day to about 10 mg/kg body weight per day, from about 3 mg/kg body weight per day to about 7 mg/kg body weight per day, from about 3 mg/kg body weight per day to about 5 mg/kg body weight per day, from about 2 mg/kg body weight per day to about 7 mg/kg body weight per day, or from about 2 mg/kg body weight per day to about 5 mg/kg body weight per day. In some embodiments, the amount is about 2, about 3, about 4, about 5, about 6, or about 7 mg/kg body weight per day. The administration may be once per day, twice per day, or more than two times per day.

Additionally, in some embodiments, a patient may receive the NBD peptide by multiple administration methods. In some embodiments, the NBD peptide may be administered to the patient by injection, nebulization, buccal administration, oral administration (e.g., solution, tablet, thin film, etc.), transdermal patch, intranasally, and any combination of the foregoing. For example, the NBD peptide may be administered to the patient intranasally in addition to an oral administration. In some embodiments, oral administration may be used to maintain an optimal drug concentration in the patient during intranasal treatment. In some embodiments, the NBD peptide may be administered to the patient intranasally in addition to injection(s). In some embodiments, the NBD peptide may be administered to the patient intranasally in addition to a transdermal patch. In some embodiments, the NBD peptide may be administered to the patient intranasally in addition to using a nebulization technique. In some embodiments, the agents are administered orally only. The present disclosure encompasses any combination of the administration techniques described or contemplated herein.

The present inventor discovered that the pharmaceutical compositions disclosed herein, along with the administration methods, can be used to improve locomotor and cognitive activities (see Examples disclosed herein). As such, the present disclosure is also directed to compositions and methods useful for improving locomotor and/or cognitive activities. In some embodiments, the locomotor activities are selected from the group consisting of walking, running, jumping, and any combination thereof.

Any or all of these locomotor activities may be improved by administering a pharmaceutical composition to a patient, wherein the composition comprises a NBD peptide. In some embodiments, the composition is administered intranasally. Depending upon the administration method and the number of administrations per day (optionally among other factors), an effective amount can be selected by one of ordinary skill in the art with the guidance provided in the present application.

Additionally, the present inventor discovered that the pharmaceutical compositions disclosed herein, along with the administration methods, can be used to reduce activation of certain cells in the brain. For example, using the pharmaceutical compositions disclosed in the present application in combination with one or more of the administration methods disclosed herein, the inventor discovered that it is possible to reduce activation of microglial cells in the brain (see Examples disclosed herein).

Still further, the inventor discovered that the presently disclosed pharmaceutical compositions and methods of administration can be used to reduce levels of α-synuclein in the brain (see Examples disclosed herein).

NF-κB essential modifier-binding domain (NBD) Proteins Useful in the Invention May et al discloses various sequences of peptides that inhibit NF-κB activation through inhibition of kB (IkB)-kinase (i.e. IKK inhibitors, IKKa and IKKb) via the regulatory protein NEMO (NF-κB essential modifier. They disclose an amino-terminal a-helical region of NEMO associated with a carboxyl-terminal segment of IKKa and IKKb that are called the NEMO-binding domain (NBD). See May et al. “Selective Inhibition of NF-κB Activation by a Peptide That Blocks the Interaction of NEMO with the IkB Kinase Complex,” (2000) Science 289: pp. 1550-1554.

The wild type NBD (wtNBD) is disclosed as:

(SEQ ID NO: 1)
TALDWSWLQTE.

To facilitate entry into cells, it can be attached to the Antennapedia homeodomain (DRQIKIWFQNRRMKWKK; see Ghosh et al. 2007 and Mondal et al. 2012). Thus, another wtNBD peptide useful in the invention would be an NBD peptide attached to the Antennapedia homeodomain as:

(SEQ ID NO: 2)
DRQIKIWFQNRRMKWKKTALDWSWLQTE.

Further, the inventor has found in previous studies that the truncated hexapeptide of the wtNBD, LDWSWL (SEQ. ID. NO: 3) is sufficient to block the function of NF-κB in cultured brain cells and in vivo in the brain. See Ghosh et al. 2007 and Mondal et al. 2012. This sequence can also be couple to the Antennapedia homeodomain. Id.

(SEQ ID NO: 4)
DRQIKIWFQNRRMKWKKLDWSWL.

Neither of the mutated forms of wtNBD are capable of blocking the function of NF-κB. See May et al. 2000, Ghosh et al. 2007, Mondal et al. 2012 and the current invention.

It can be easily recognized that any of the peptides of SEQ ID NO: 1, SEQ ID NO: 2, SEQ ID NO: 3, and SEQ ID NO: 4 are useful as wtNBD peptides capable of blocking the function of NF-κB in the compositions and methods of the current invention.

Further reference is made to the following experimental examples.

### EXAMPLES

The following examples are given for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

Intranasal Treatment of Animals with wtNBD Peptide

The wtNBD peptide (SEQ ID NO: 4) was solubilized in normal saline in such a way so that each mouse receives 0.1 mg/kg of body weight NBD peptide in 2.5 μl of saline. Then 2.5 μl of wtNBD solution was administered in mice through each nostril every day for a total of 30 days. Mice were hold in supine condition while administering wtNBD solution. See Rangasamy et al. “Selective disruption of TLR2-MyD88 interaction inhibits inflammation and attenuates Alzheimer's pathology” (2018) J Clin Invest 128, 4297-4312. Intranasal treatment was started for preformed α-syn fibril (PFF) or PBS injected A53T animals at the age of 5 months (2 months following the brain surgery) for the next 30 days. Aged A53T animals (8 months old) were also treated with wtNBD peptides for 1 month followed by behavioural tests and other experiments at the age of 9 months.

### Example 2

Intranasal Administration of NEMO-Binding Domain (NBD) Peptide (SEQ ID NO: 4).

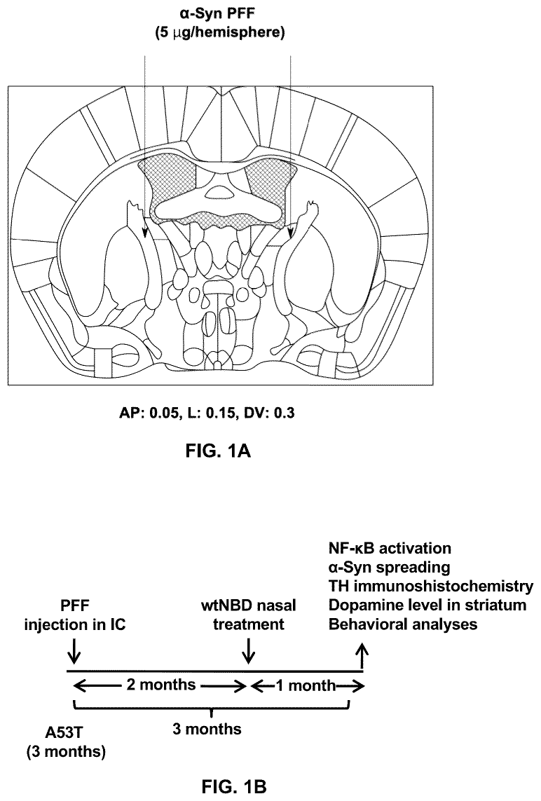

It was surprisingly found that intranasal administration of the NBD peptide (SEQ ID NO: 4) reduces α-syn spreading from striatum to nigra in PFF-seeded mice. Preformed α-syn fibril (PFF) was injected into the internal capsule (IC) region of striatum in both hemispheres of the mice (FIG. 1A), and following 2 months of PFF seeding, animals received nasal delivery of 0.1 mg/kg/d of wtNBD peptide for the next 1 month. The experimental animals were sacrificed at the age of 6 months and several biochemical tests were conducted to find out the effect of wtNBD treatment on PFF-induced pathology (FIG. 1B).

Since microglial activation plays an important role in different neurodegenerative diseases and activation of NF-κB is needed for microglial inflammation, the role of NF-κB in α-syn spreading and associated pathology in the brain was investigated. The wtNBD peptide is a specific inhibitor of NF-κB activation (May et al., 2000) and it has been previously demonstrated that after intranasal administration, wtNBD peptide enters into the brain. See Rangasamy et al. “Intranasal Delivery of NEMO-Binding Domain Peptide Prevents Memory Loss in a Mouse Model of Alzheimer's Disease, (2015) J Alzheimers Dis 47, 385-402. Therefore, the effect of intranasal wtNBD on α-syn spreading in the brain was examined.

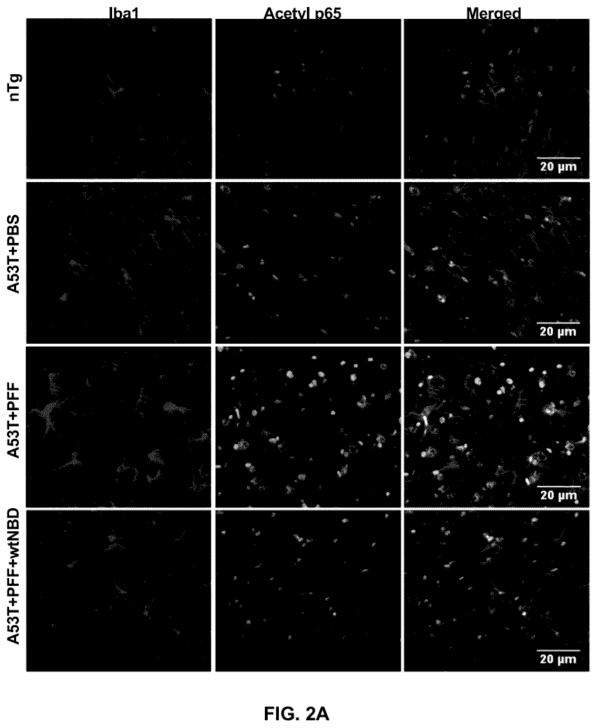

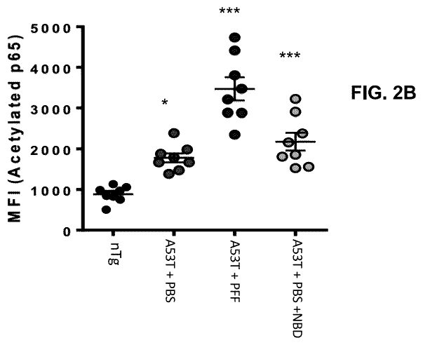

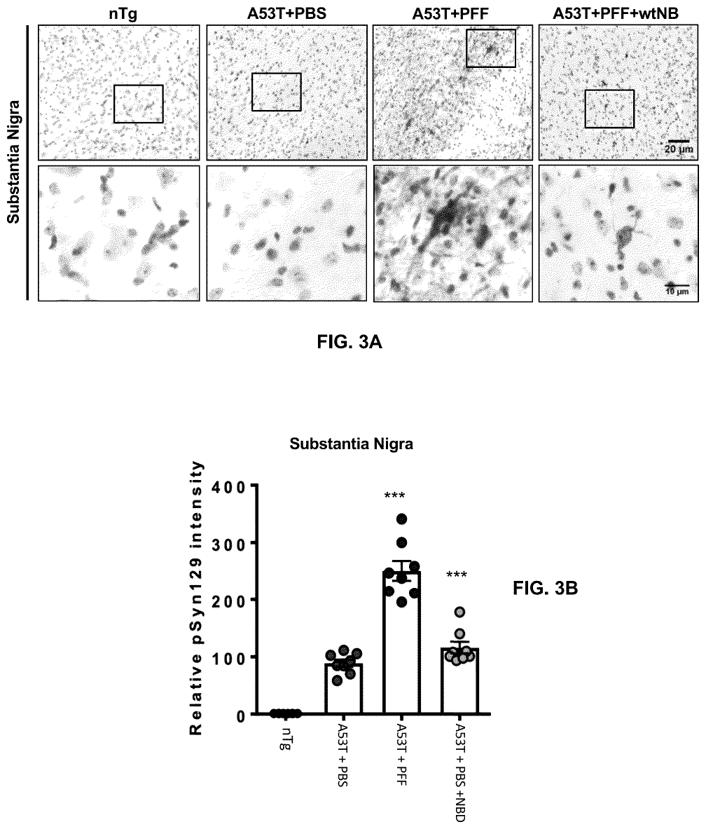

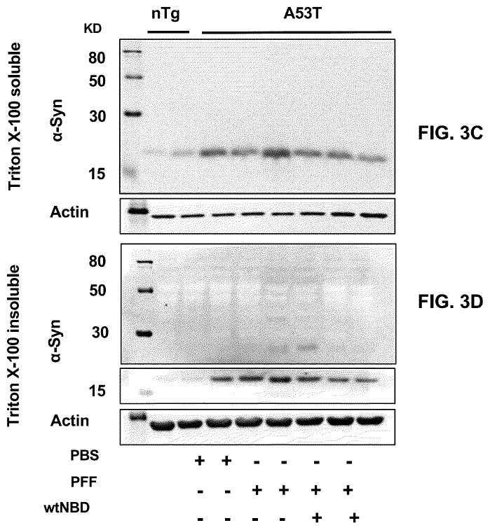

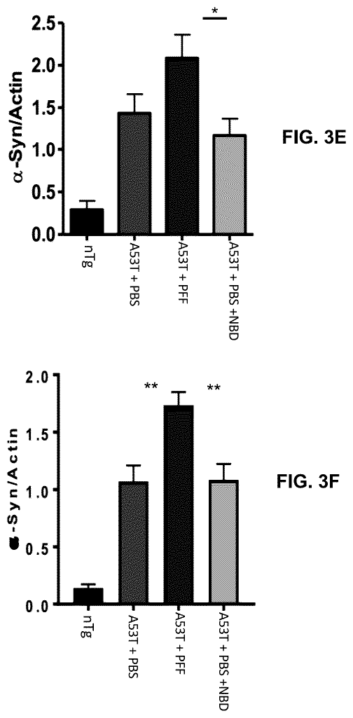

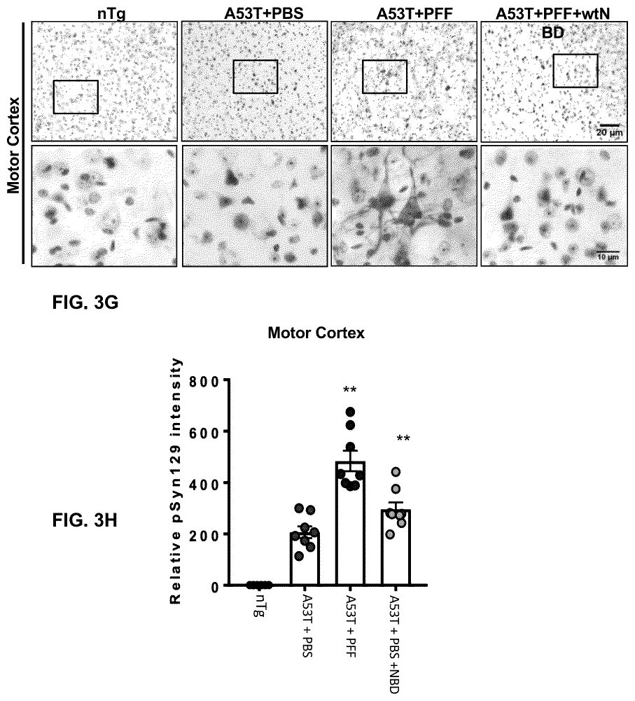

Following 1 month of wtNBD administration, NF-κB activation in substantia nigra (SN) and spreading of α-syn in both SN and motor cortex were monitored. Marked up-regulation of microglial acetylated p65 level in the SN of PFF-seeded mice was found compared to the PBS-injected mice (FIG. 2A and FIG. 2B). However, acetylated p65 level significantly decreased in wtNBD-treated mice brain (FIG. 2A and FIG. 2B). PFF-seeding resulted in exaggerated accumulation of pSyn129 in nigral neurons (FIG. 3A and FIG. 3B). However, intranasal wtNBD peptide drastically reduced the level of pSyn129 in these neurons, which is reflected by the relative optical density measurement of pSyn129 in SN (FIG. 3A and FIG. 3B). This observation was also verified by immunoblotting, where PFF-seeded mice exhibited the presence of higher level of detergent insoluble form of α-syn than PBS-injected mice (FIG. 3D and FIG. 3F). However, following wtNBD treatment α-syn contents in both soluble and insoluble fractions were significantly reduced (FIG. 3C—FIG. 3F). Similar to the nigra, wtNBD peptide also reduced the spreading of α-syn in motor cortex as evidenced by reduced accumulation of pSyn129 in cortical neurons of wtNBD-treated PFF-seeded mice as compared to saline-treated PFF-seeded mice (FIG. 3G and FIG. 3H).

### Example 3

Intranasal NBD Peptide (SEQ ID NO: 4) Protects Dopaminergic Neurons and Improves Locomotor Activities in PFF-Seeded Mice

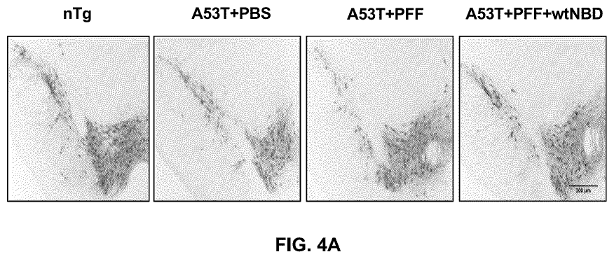

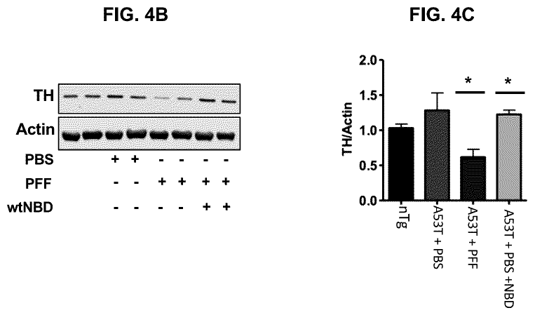

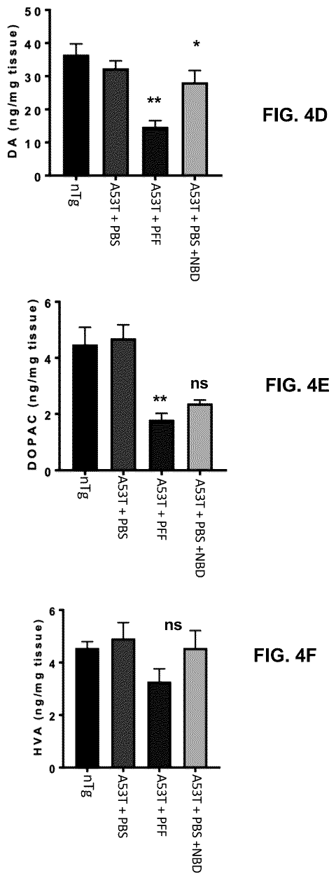

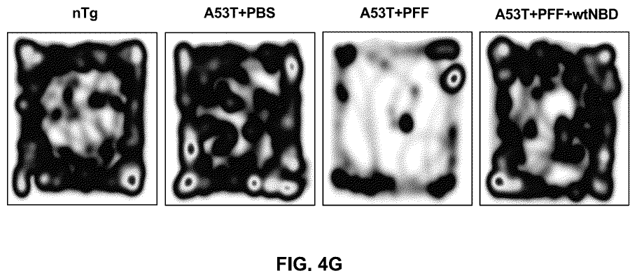

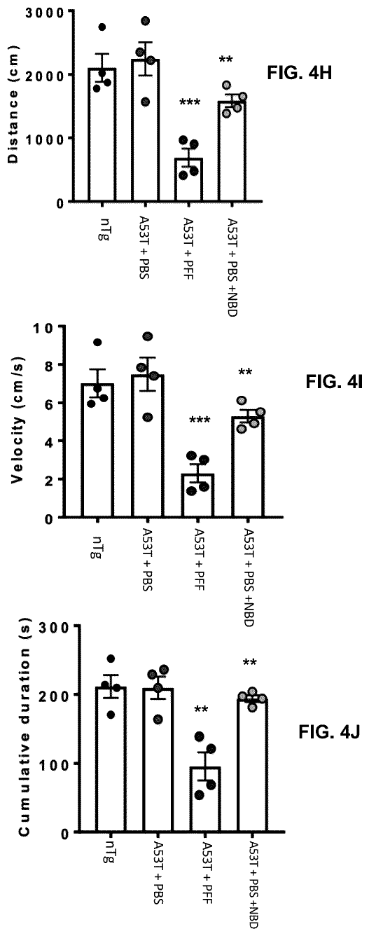

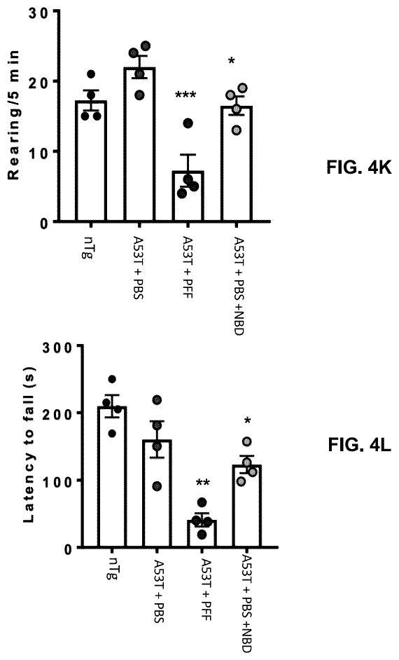

Next, the effect of intranasal wtNBD on PFF-induced Parkinsonian pathologies was monitored. Significantly reduced number of TH neurons (FIG. 4A) as well as nigral TH protein level (FIG. 4B and FIG. 4C) were found in PFF-seeded mice than the PBS-injected group. Demise of nigral TH neurons resulted in depletion of neurotransmitters in the striatum of PFF-seeded animals (FIG. 4D-FIG. 4F). Interestingly, nigral TH neurons, TH protein level and the striatal DA level were significantly protected in wtNBD-treated mice (FIG. 4A-FIG. 4F). As expected, PFF-seeding also resulted in deficit in locomotor activities of A53T animals (FIG. 4G) as demonstrated by open field test parameters such as distance (FIG. 4H), velocity (FIG. 4I), cumulative duration of movement (FIG. 4J), and rearing (FIG. 4K). Locomotor deficit was also evident by rotarod analysis (FIG. 4L). However, concomitant with dopaminergic neuronal protection, wtNBD treatment significantly inhibited the movement deficits in PFF-seeded mice (FIG. 4G-FIG. 4L). These results suggest that intranasal wtNBD peptide was capable of protecting dopaminergic neurons and improving locomotor activities in PFF-seeded mice.

As will be appreciated from the descriptions herein, a wide variety of aspects and embodiments are contemplated by the present disclosure, examples of which include, without limitation, the aspects and embodiments listed below:
Methods and pharmaceutical compositions and/or formulations useful for the treatment of neurodegenerative and disorders involving α-synucleinopathy;
Methods and pharmaceutical compositions and/or formulations comprising agents that inhibit NF-κB activation;
Methods and pharmaceutical compositions comprising a NEMO-binding domain (NBD) peptide to slow or inhibit the progression of neurodegenerative and disorders involving α-synucleinopathy;
Pharmaceutical compositions that comprise an agent that inhibits NF-κB activation where the agent is a wild-type NEMO-binding domain (wtNBD) peptide;
Pharmaceutical compositions that comprise an agent that inhibits NF-κB activation where the agent is a wild-type NEMO-binding domain (wtNBD) peptide where the wtNBD peptide contains the Antennapedia homeodomain or similar peptide sequence to promote entrance into the cells.

In any embodiments, the pharmaceutical composition is formulated together with a pharmaceutically acceptable carrier or excipient;

In still other embodiments, the pharmaceutical composition is preferably administered intranasally;

Pharmaceutical compositions that are used to treat or inhibit the progression or spreading of α-syn, more particularly, disorders that involve α-synucleinopathy;

Pharmaceutical compositions are used to treat or inhibit the progression or spreading of α-syn in disorders including multiple system atrophy (MSA), dementia with Lewy bodies (DLB), PD, multiple sclerosis (MS), optic neuritis (ON), Huntington disease (HD), Amyotrophic lateral sclerosis (ALS), or any disorder in which microglial activation may play a role in disease pathogenesis;

Methods and pharmaceutical compositions comprising a NEMO-binding domain (NBD) peptide where the NBD peptide is a wild-type NBD peptide (wtNBD);

Methods and pharmaceutical compositions wtNBD comprises any one or more of SEQ ID NO: 1, SEQ ID NO: 2, SEQ ID NO: 3, and SEQ ID NO: 4.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for slowing or inhibiting the progression of an α-synucleinopathy disorder or a microglial activation disorder in a subject, the method comprising administering to the subject in need of such treatment a therapeutically effective amount of a pharmaceutical composition comprising NF-κB essential modifier (NEMO)-binding domain (NBD) peptide.

2. The method of claim 1, wherein the NBD peptide is a wild-type NEMO-binding domain (wtNBD) peptide.

3. The method of claim 1, wherein the NBD peptide is selected from the group consisting of: SEQ ID NO: 1, SEQ ID NO: 2, SEQ ID NO: 3, and SEQ ID NO: 4.

4. The method of claim 1, wherein the NBD peptide contains a peptide sequence to promote entrance into a cell.

5. The method of claim 4, wherein the peptide sequence to promote entrance into a cell is the Antennapedia homeodomain sequence DRQIKIWFQNRRMKWKK.

6. The method of claim 1, wherein the pharmaceutical composition is formulated together with a pharmaceutically acceptable carrier or excipient.

7. The method of claim 1, wherein the pharmaceutical composition is administered intranasally.

8. The method of claim 1, wherein the α-synucleinopathy disorder or a microglial activation disorder is selected from the group consisting of multiple system atrophy (MSA), dementia with Lewy bodies (DLB), PD, multiple sclerosis (MS), optic neuritis (ON), Huntington disease (HD), and Amyotrophic lateral sclerosis (ALS).

9. A pharmaceutical composition for slowing or inhibiting the progression of an α-synucleinopathy disorder or a microglial activation disorder in a subject in need of such treatment, wherein the pharmaceutical composition comprises a therapeutically effective amount of an agent that inhibits NF-κB activation, wherein the agent that inhibits NF-κB activation is a wild-type NEMO-binding domain (wtNBD) peptide.

10. The pharmaceutical composition of claim 9, wherein the agent that inhibits NF-κB activation is a wild-type NEMO-binding domain (wtNBD) peptide, wherein wtNBD peptide is selected from the group consisting of: SEQ ID NO: 1, SEQ ID NO: 2, SEQ ID NO: 3, and SEQ ID NO: 4.

11. The pharmaceutical composition of claim 9, wherein the wtNBD peptide contains a peptide sequence to promote entrance into a cell.

12. The pharmaceutical composition of claim 11, wherein the peptide sequence to promote entrance into a cell is the Antennapedia homeodomain sequence DRQIKIWFQNRRMKWKK.

13. The pharmaceutical composition of claim 9 further formulated together with a pharmaceutically acceptable carrier or excipient.

14. The pharmaceutical composition of claim 9, wherein the α-synucleinopathy disorder or a microglial activation disorder is selected from the group consisting of multiple system atrophy (MSA), dementia with Lewy bodies (DLB), PD, multiple sclerosis (MS), optic neuritis (ON), Huntington disease (HD), and Amyotrophic lateral sclerosis (ALS).

15. The pharmaceutical composition of claim 9, wherein the pharmaceutical composition is administered intranasally.
